(19) United States
(12) Patent Application Publication
(10) Pub. No.: US 2026/0047962 A1
(43) Pub. Date: Feb. 19, 2026

(54) TAMPON WITH INTEGRATED DEVICE FOR IMPROVED INSERTION, AND REMOVAL, AND LEAK PREVENTION

(71) Applicant: Lauren Clark, Sebastopol, CA (US)

(72) Inventor: Lauren Clark, Sebastopol, CA (US)

(21) Appl. No.: 19/303,784

(22) Filed: Aug. 19, 2025

Related U.S. Application Data
(60) Provisional application No. 63/684,538, filed on Aug. 19, 2024.

(51) Int. Cl. A61F 13/26; A61F 13/20; A61F 13/34

(52) U.S. Cl. CPC A61F 13/263; A61F 13/2037; A61F 13/2071; A61F 13/208; A61F 13/2097; A61F 13/34

(57) ABSTRACT

The present invention introduces a tampon with an integrated device designed for improved insertion, and removal, and leak prevention. The device comprises a flexible, nonpermeable wall, hollow to enable finger-assisted insertion and featuring a removal aid element attached to the bottom end of the wall for easy, hygienic extraction. Additionally, the wall is equipped with ridges that conform to the vaginal wall, enhancing the seal to prevent menstrual fluid leakage and block the absorption of external fluids. In other embodiments, a smoothed wall is offered. This invention eliminates the need for separate applicators, allows the device to compact for discrete portability, and significantly enhances user comfort and effectiveness in fluid management.

### CROSS-REFERENCE TO RELATED APPLICATIONS

This application claims the benefit of U.S. Provisional Application No. 63/684,538, filed Aug. 19, 2024, the entire disclosure of which is hereby incorporated by reference.

### BACKGROUND OF THE INVENTION

### 1. Field of the Invention The present invention relates to personal hygiene products, but more particularly a tampon with integrated device for improved insertion, and removal, and leak prevention.

### 2. Description of Related Art

Traditional tampons are primarily designed for absorbing menstrual fluids but often require separate applicators for insertion and do not adequately address leakage prevention or the entry of external fluids. Common issues with existing tampons include discomfort during insertion and removal, potential for leakage, and the necessity for frequent changes due to saturation or external fluid contamination. Furthermore, most tampons come with applicators, which can be cumbersome to use and environmentally wasteful. Consequently, a solution is provided herein.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

It is an object of the invention to provide a tampon device that simplifies the insertion and removal process, and enhances comfort during use, and provides superior protection against leaks and external fluid contamination.

The foregoing has outlined rather broadly the more pertinent and important features of the present invention so that the detailed description of the invention that follows may be better understood and so that the present contribution to the art can be more fully appreciated. Additional features of the invention, which will be described hereinafter, form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF THE DRAWINGS

Other features and advantages of the present invention will become apparent when the following detailed description is read in conjunction with the accompanying drawings, in which:

FIG. 1 is a front view of the tampon with integrated device according to an embodiment of the present invention.

FIG. 2 is a front sectional view of the tampon with integrated device with a finger illustrating operation according to an embodiment of the present invention.

FIG. 3 is a detailed view illustrating the folding capability of the tampon with integrated device according to an embodiment of the present invention.

FIG. 4 illustrates a sectional diagram view showing the tampon with integrated device position during use according to an embodiment of the present invention.

FIG. 5 is a first alternate tampon with integrated device without ridges according to an embodiment of the present invention.

FIG. 6 shows several views of a second alternate tampon having an integrated loop structure within the device and one example of an attachment method via stitching according to an embodiment of the present invention.

FIG. 7 shows several views of a third alternate tampon having an integrated loop structure within the device and another example of an attachment method via stitching according to an embodiment of the present invention.

FIG. 8 shows several views of a fourth alternate tampon according to an embodiment of the present invention.

FIG. 9 shows several views of a fifth alternate tampon according to an embodiment of the present invention.

FIG. 10 shows several views of a sixth alternate tampon with and another example of an attachment method via stitching according to an embodiment of the present invention.

FIG. 11 shows several views of a seventh alternate tampon having an integrated loop structure within the device and another example of an attachment method via stitching according to an embodiment of the present invention.

### DETAILED DESCRIPTION OF THE INVENTION

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide a tampon with integrated device for improved insertion, and removal, and leak prevention.

It is to be understood that the terminology used herein is for the purpose of describing particular embodiments only and is not intended to be limiting. The terms “a” or “an,” as used herein, are defined to mean “at least one. ” The term “plurality,” as used herein, is defined as two or more. The term “another,” as used herein, is defined as at least a second or more. The terms “including” and/or “having,” as used herein, are defined as comprising (i.e., open language). The term “providing” is defined herein in its broadest sense, e.g., bringing/coming into physical existence, making available, and/or supplying to someone or something, in whole or in multiple parts at once or over a period of time. These terms generally refer to a range of numbers that one of skill in the art would consider equivalent to the recited values (i.e., having the same function or result). In many instances these terms may include numbers that are rounded to the nearest significant figure.

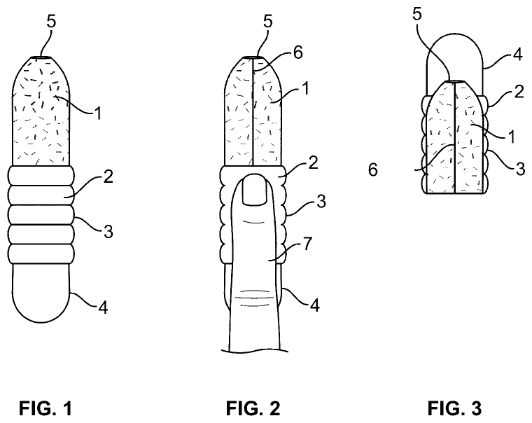

FIG. 1 illustrates a front view of the tampon with integrated device according to an embodiment of the present invention. In one embodiment, the device includes a tampon (1) having an absorbent body designed for managing menstrual fluids effectively. In some embodiments, the absorbent body is constructed from a selection of materials that can include cotton, rayon, sponge, and bamboo fiber. These materials may be used in their organic form, synthetic equivalents, or as blends to enhance the properties of the tampon such as absorbency, comfort, and environmental sustainability. As understood in the art, the non-limited material choice allows the tampon to be tailored to meet diverse user needs and preferences while maintaining high performance in fluid absorption.

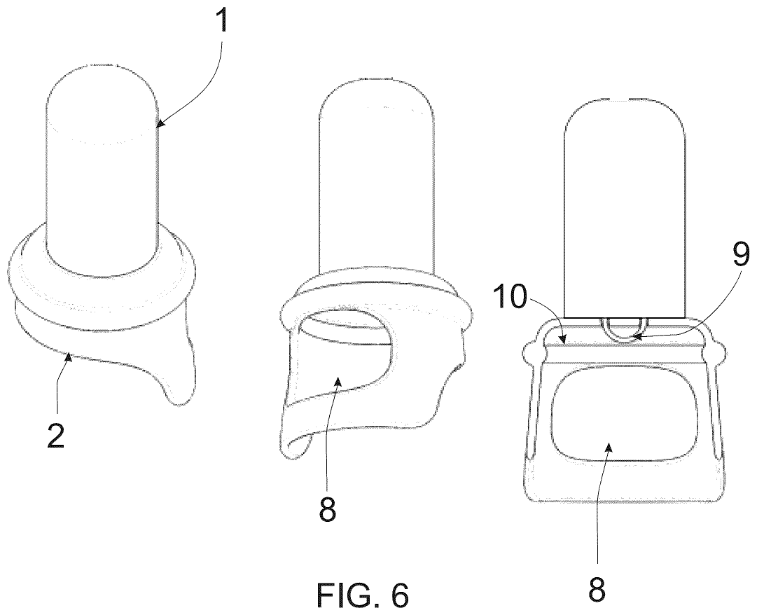

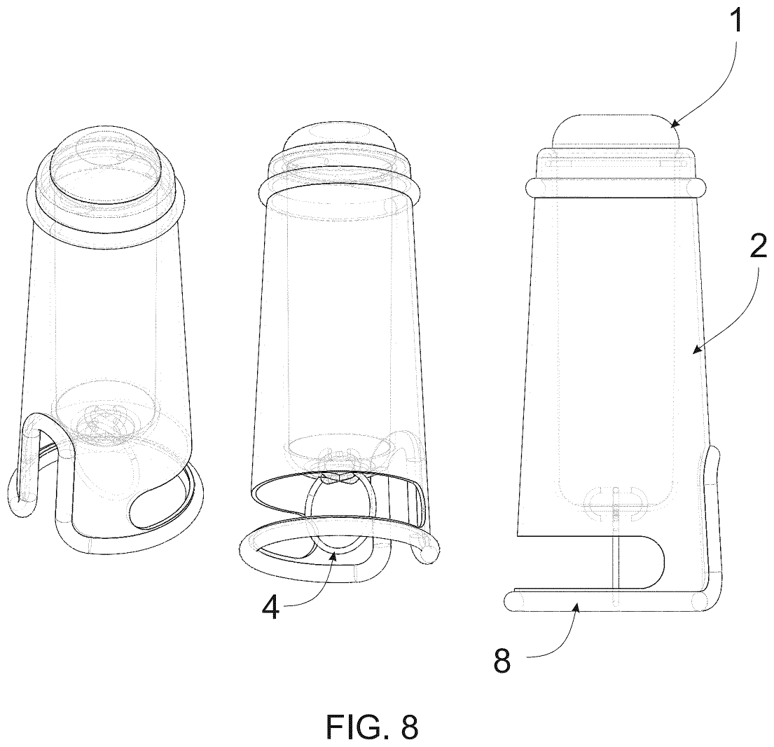

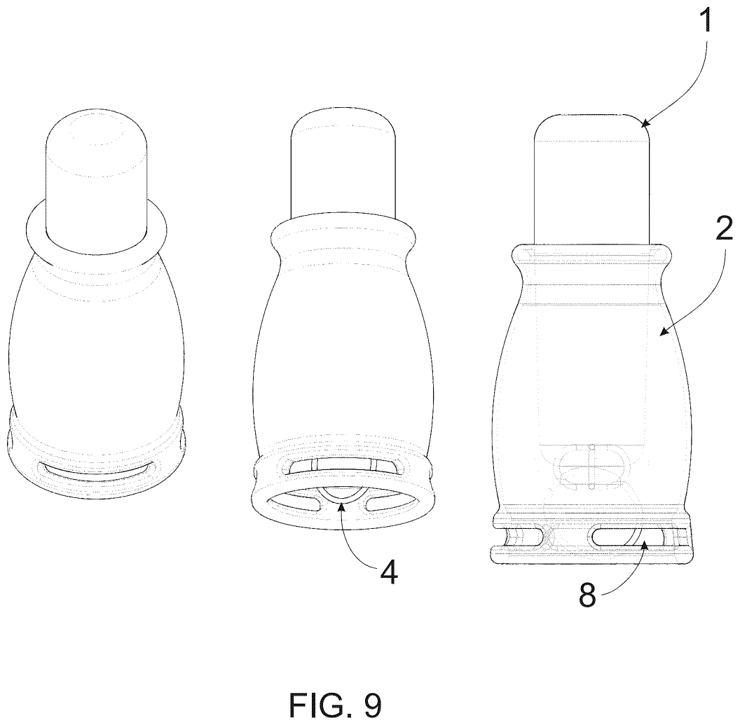

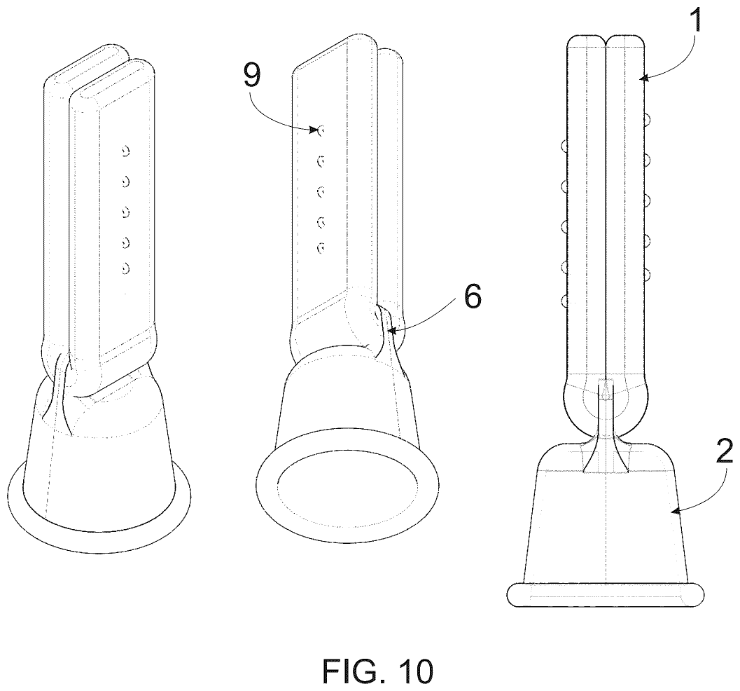

In one embodiment, attached to the bottom side of the tampon (1) is a wall (2), a flexible, nonpermeable structure with a hollow center that serves as both an insertion aid, a removal aid, and a seal against the vaginal wall. Advantageously, the wall (2) is designed to maintain its structural integrity while conforming to the vaginal wall to prevent leakage and block the absorption of external fluids into the tampon. In one embodiment, the wall (2) has one or more ridges (3) that run circumferentially on the exterior of the wall (2) which are configured to abut the vaginal wall when the device is in use and enhance the seal between the device and the vaginal wall to prevent leaks, providing additional protection and comfort. In one embodiment, seen in FIG. 10, the seal is made by a ridge at the bottom of the wall (2). In one embodiment, the wall (2) has a top side that meets the bottom side of the tampon (1) and completes a fluid-proof seal between the tampon and the external environment to prevent menstrual fluids from leaking out the hollow center of the wall (2) and to prevent external fluids from entering the tampon (1) through the hollow center of the wall (2). In one embodiment, the tampon (1) is attached to the top side of the wall (2) by adhesive. In another embodiment, the tampon (1) is attached to the top side of the wall (2) using stitching (FIG. 6; 9). In one embodiment, the wall (2) has a flap (FIG. 6; 10) to cover the portion of the wall with the stitching (FIG. 6; 9) to ensure a fluid-proof seal around the stitching. In some embodiments, seen in FIGS. 8-9, a significant portion of the tampon (1) sits inside the hollow center of the wall (2) and only a portion of the tampon (1) extends above the top end of the wall (2). The wall extending up over the tampon has the advantage of easier insertion and removal, as it acts like a built-in applicator to prevent the tampon from dragging along the vaginal canal causing discomfort. Further, this configuration adds additional leak prevention because it covers more of the tampon and only allows blood to enter from the top. In some embodiments, the wall may be made of plastic, rubber, silicone or any other material suited for its purpose. The wall may be different lengths, shapes and sizes with varying numbers and shapes of ridges without departing from the spirit of the invention.

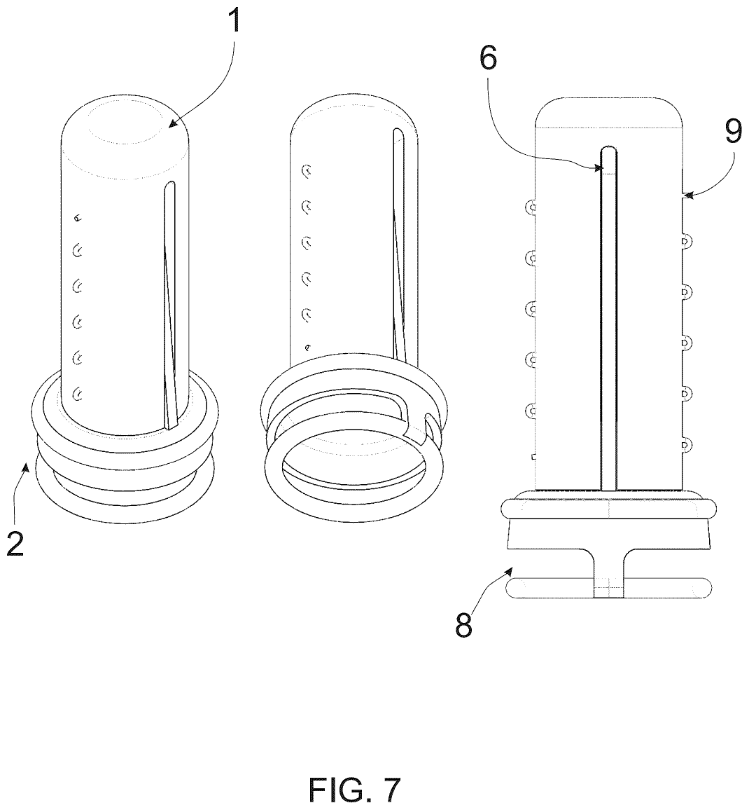

In one embodiment, the device has a removal aid element (4) below the wall (2), which facilitates easy and hygienic removal of the device from within the vagina allowing the user to pull the tampon out of the vagina without direct contact with absorbed fluids. The removal aid element (4) is also visible and easy to find, which helps to prevent the tampon device from getting lost inside the vagina which is a common problem identified with the prior art. In one embodiment, the removal aid element (4) is a pull string connected at two points to the bottom edge of the wall (2) so that it hangs down from the device in a U-shape as seen in FIG. 1. In another embodiment, seen in FIGS. 6-9, 11 the removal aid is integrated into the wall (2) as a loop structure (8) at the bottom end of the wall (2). In another embodiment the device has a loop structure (8) at the bottom end of the wall as well as a pull string removal aid (4).

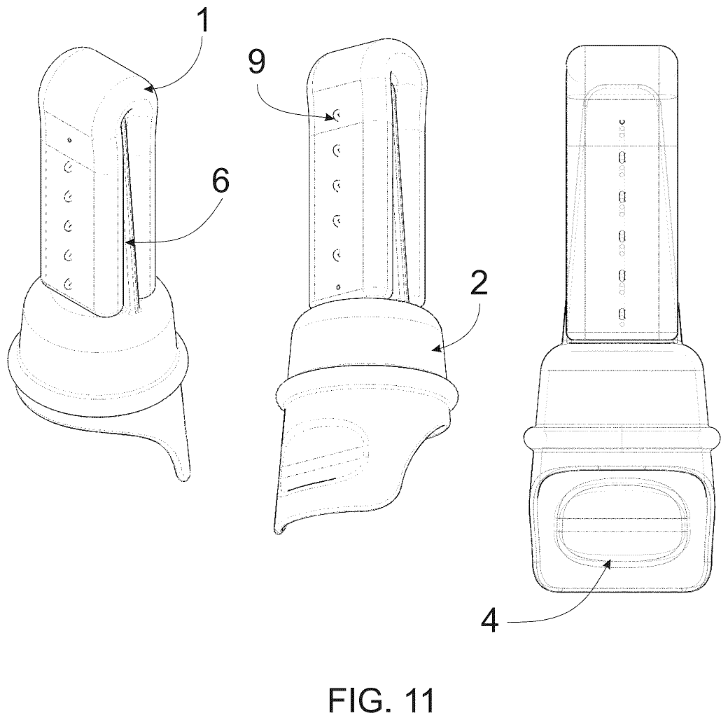

In some embodiments, an attachment cap (5) is provided at the device's proximal or top end, which is at the top end of the tampon (1) and wherein the attachment cap (5) is connected to an attachment stick (6) which is connected to the base of the tampon (1) and wall (2) connection. In one embodiment, the attachment stick (6) is attached to the top side of the wall (2). It may be attached by varying means without departing from the spirit of the invention. In some embodiments, the attachment stick is attached to the wall by stitching and in other embodiments it is attached by adhesive. In some embodiments, the attachment stick is integral to the wall. The attachment cap (5) and attachment stick (6) together serve to provide additional rigidity to the tampon to help keep it intact during insertion, use and removal. The attachment stick (6) may have varying shapes and sizes as described in multiple embodiments here and seen in the figures. Additional shapes and sizes may be envisioned without departing from the spirit of the invention. In one embodiment, the attachment cap (5) and attachment stick (6) serve to attach the tampon (1) to the wall (2). In one embodiment, the tampon (1) is attached to the attachment cap (5) or attachment stick (6) by stitching (9) or adhesive. In one embodiment, the attachment stick (6) runs through the middle of the tampon (1) so that it is surrounded by tampon material on all sides. In another embodiment, seen in FIG. 11, the tampon (1) is folded over the top of the attachment stick (6) and attached to the attachment stick (6) with stitching (9). In another embodiment, seen in FIG. 10, the attachment stick is an attachment loop (6) and the tampon (1) passes through the attachment loop (6) and is folded in half and the two halves of the tampon (1) are attached together by stitching (9) to hold the tampon in place within the attachment loop (6). The attachment cap (5) and attachment stick (6) may be made of the same material as the wall (2) or they may be made of different materials including plastic, rubber, silicone or any other material suited for its purpose. The stitching (9) may be made of cotton thread, synthetic thread, textile mesh fabrics or any other material suited for its purpose.

FIG. 2 shows a front sectional view of the tampon with integrated device with a finger (7) of a user demonstrating the operation according to an embodiment of the present invention. The hollow interior of the wall (2) allows for the insertion of a finger (7), facilitating the placement of the tampon (1) into the vagina using the finger (7) to push the device into the vagina. Advantageously, this design enhances user comfort and ensures correct positioning without the need for an applicator.

FIG. 3 provides a detailed view illustrating the folding capability of the device according to an embodiment of the present invention. In one embodiment, the device folds in half at the connection point between the tampon (1) and the wall (2), which compacts the total size of the device during storage and transportation, such as in a purse or handbag. In one embodiment, the wall (2) is able to compress and lay flat as a result of its flexible construction and hollow core, while the tampon (1) is able to compress and flatten as due to its material construction. In one embodiment, the device resists folding and remains in an extended position. In another embodiment, the wall (2) is configured to fold allowing the tampon (1) to be pushed down into the hollow center of the wall (2) in order to shrink the size of the device and to protect the tampon (1). In another embodiment, the length of the wall (2) is much less than the length of the tampon (1) and thus the overall length and size of the device is reduced and thus folding is not necessary.

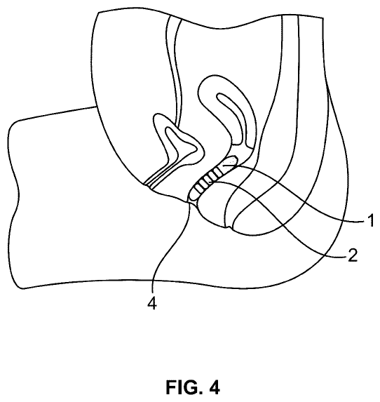

FIG. 4 illustrates a sectional diagram view showing the device positioned within a vagina during use according to an embodiment of the present invention. The device is inserted with the tampon (1) end going in first and the wall (2) below it and the wall (2) used as an insertion aid. The ridges (3) on the wall (2) press against the vaginal wall, enhancing the seal and preventing leaks, thereby providing superior protection and comfort while in use. The removal aid element (4) is positioned near the vaginal opening to allow easy access to the user for finding and removing the tampon device.

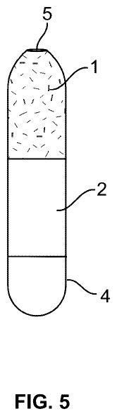

FIG. 5 depicts an alternate embodiment of the device with a smooth wall (2) void of ridges according to an embodiment of the present invention. This embodiment is designed for users who prefer a smoother interface with the vaginal wall. Despite lacking ridges (3), it retains the beneficial features of the wall (2) and the removal aid element (4) for ease of insertion and removal, as well as effective leak prevention with the smooth wall (2) still making a seal with the vaginal wall.

The process for insertion and removal of the device from the vagina will now be described. First, the device is inserted into the vagina with the tampon (1) end of the device entering first. A user's finger is inserted into the hollow center of the wall (2) and used to push the device all the way into the vagina to create a seal between the device wall (2) and the vaginal wall to prevent leakage of fluids. The removal aid element (4) hangs from the bottom of the wall (2) and is visible or easily accessible to the user at the vaginal opening. The device is left inside of the vagina for an extended period of time to absorb menstrual fluids. When the user is ready to remove the device, they pull the device out by pulling on the removal aid element (4). When the removal aid (4) in turn pulls on the wall (2) it causes the wall (2) to deform and the seal between the wall (2) and the vaginal wall to break which allows the device to come out easily. Alternatively, the device may be configured to be removed by pulling directly on the wall (2).

Although the invention has been described in considerable detail in language specific to structural features, it is to be understood that the invention defined in the appended claims is not necessarily limited to the specific features described. Rather, the specific features are disclosed as exemplary forms of implementing the claimed invention. In other words, the terminology and phraseology used in this description and the abstract are for illustrative purposes and should not be considered limiting.

Therefore, while exemplary illustrative embodiments of the invention have been described, numerous variations and alternative embodiments will occur to those skilled in the art. Such variations and alternative embodiments are contemplated, and can be made without departing from the spirit and scope of the invention.

It should further be noted that throughout the entire disclosure, the labels such as left, right, front, back, top, bottom, forward, reverse, clockwise, counterclockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, references to “first,” “second,” “third,” and so forth members throughout the disclosure (and in particular, claims) are not used to show a serial or numerical limitation but instead are used to distinguish or identify the various members of the group.

1. A tampon device comprising:
a tampon;
a nonpermeable wall attached to a bottom end of the tampon;
a removal aid element attached to a bottom end of the wall;
the nonpermeable wall configured to form a seal with the vaginal wall when inserted into the vagina; and,
the nonpermeable wall being hollow to allow for the insertion of a finger of a user into the nonpermeable wall for applying pressure to the bottom side of the tampon for the purpose of inserting the tampon device into the vagina.

2. The tampon device of claim 1, wherein the nonpermeable wall attached to the bottom end of the tampon has one or more ridges that run circumferentially around the nonpermeable wall configured to enhance the seal between the nonpermeable wall and the vaginal wall.

3. The tampon device of claim 1, wherein the removal aid element is a pull string attached at two points to the bottom side of the nonpermeable wall and hanging down from the nonpermeable wall in a U-shape.

4. The tampon device of claim 1, wherein the removal aid element is integral to the nonpermeable wall and is a loop portion at the bottom end of the nonpermeable wall.

5. The tampon device of claim 1, wherein the nonpermeable wall has a top side that meets the bottom side of the tampon and prevents movement of fluid between the tampon and the external environment.

6. The tampon device of claim 1, wherein the tampon is attached to a top side of the nonpermeable wall with stitching and a flap on the nonpermeable wall covers the stitching to provide a fluid-proof seal for the stitching.

7. The tampon device of claim 1, further comprising an attachment cap at the top end of the tampon connected to an attachment stick running longitudinally down the length of the tampon and connected to the top side of the nonpermeable wall, and the attachment cap and attachment stick together providing additional rigidity to the tampon to aid in insertion of the tampon device.

8. The tampon device of claim 1, wherein the nonpermeable wall has a smooth outer surface void of ridges and configured to create a seal between the nonpermeable wall and the vaginal wall.

9. A method for using a tampon device, comprising:
inserting a finger of a user into the bottom opening of a nonpermeable wall attached at a top side of the nonpermeable cylindrical wall to a bottom side of a tampon of a tampon device;
inserting the tampon device into a vagina of a user with the tampon side of the tampon device entering first;
applying pressure on the tampon with the finger inserted into the bottom opening of the nonpermeable wall to push the tampon into place fully inside the vagina with the nonpermeable wall forming a seal with the vaginal wall;
leaving the tampon device inside the vagina for an extended period of time; and,
removing the tampon device from the vagina by pulling on a removal aid element attached to the bottom side of the nonpermeable wall.
